(19) United States
(12) Patent Application Publication
(10) Pub. No.: US 2004/0166126 A1
(43) Pub. Date: Aug. 26, 2004

(54) Heat activated durable styling compositions comprising aminated C5 to C7 saccharide unit and methods for same

(76) Inventors: David W. Cannell, Plainfield, NJ (US); Nghi Van Nguyen, Edison, NJ (US)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 09/820,648

(22) Filed: Mar. 30, 2001

(51) Int. Cl. A61K 6/00

(52) U.S. Cl. 424/401

(57) ABSTRACT

Compositions, optionally heat-activated, methods and kits for durable non-permanent shaping of at least one keratinous fiber or for durable retention of a non-permanent shape of at least one keratinous fiber comprising applying to keratinous fibers a composition comprising at least one compound comprising at least one C₅ to C₇ saccharide unit substituted with at least one amino group, and heating the keratinous fibers.

The present invention relates to compositions, kits comprising these compositions, and methods for using these compositions for durable non-permanent shaping or for durable retention of a non-permanent shape of at least one keratinous fiber, including human keratinous fibers, by applying to the at least one keratinous fiber compositions which comprise at least one compound comprising at least one C₅ to C₇ saccharide unit substituted with at least one amino group, and, in certain embodiments, at least one film forming agent different from the at least one compound comprising at least one C₅ to C₇ saccharide unit substituted with at least one amino group, and heating the at least one keratinous fiber. These compositions may both impart a durable non-permanent shape to the at least one keratinous fiber and durably retain a non-permanent shape of the at least one keratinous fiber.

In today's market, many consumers prefer the flexibility of non-permanent hairstyles, that is, those styles obtained via non-permanent shaping of the hair. Typically, such non-permanent styles disappear when the hair is wetted, especially when the hair is washed with water and/or shampoo. Methods for non-permanent shaping of keratinous fibers include, for example, brushing, teasing, braiding, the use of hair rollers, and heat styling, optionally with a commercially available styling product. Non-limiting examples of heat styling include blow drying, crimping and curling methods using elevated temperatures (such as, for example, setting hair in curlers and heating, and curling with a curling iron and/or hot rollers).

While such compositions and methods may provide for non-permanent shaping of keratinous fibers, many consumers desire a higher degree of styling than most commercially available products and methods employing these products provide. For example, many consumers desire compositions and methods that improve non-permanent curl formation. There is a need, therefore, for compositions and methods for non-permanent shaping of keratinous fibers that result in a higher degree of styling, such as non-permanent curl formation.

Further, many people desire compositions and methods for retaining a particular non-permanent shape or style of keratinous fibers such as hair. A common way to retain a particular hairstyle is with the use of a hairspray, typically applied after styling the hair. Other methods to retain a hairstyle or shape of keratinous fibers include the use of mousses, gels, and lotions. The materials in these compositions are generally film forming agents, resins, gums, and/or adhesive polymers.

While such compositions and methods may provide for non-permanent shaping of keratinous fibers, many consumers desire compositions and methods for durable retention of a particular non-permanent shape or style of keratinous fibers such as hair, such as, for example, those that hold or maintain a shape of a keratinous fiber until the keratinous fiber is washed with water and/or shampoo. Further, many consumers desire compositions and methods that allow hair to retain a particular shape longer than untreated hair, even after washing or shampooing the hair.

Thus, while commercially available compositions may provide temporary setting benefits, many consumers desire a higher level of retention or hold. Good holding power is one attribute a consumer looks for in styling products for keratinous fibers. Specifically, curl retention under conditions of changing humidity, for example changes to a higher humidity, is sought after by the consumer. Further, good curl retention in damaged hair is important to the consumer since the hair fiber has been weakened and will be less likely to maintain the curl. Therefore, there is also a need for methods for durably retaining a shape of keratinous fibers even under conditions of high humidity, such as at atmospheric humidity above 40%.

Sugars and sugar derivatives are one class of the countless number of compounds that have been added to hair care compositions. Documented uses of sugars in hair care compositions include: the use of glucose to improve the tactile and elastic properties of natural hair (Hollenberg and Mueller, SOFW J. 121(2) (1995)); the use of glucose for hair damage prophylaxis and damaged hair repair (Hollenberg & Matzik, Seifen, Oele, Fette, Wachase 117(1) (1991)); the use of glucose in shampoos (J04266812, assigned to Lion Corp.); the use of trehalose for moisture retention (J06122614, assigned to Shiseido Co. Ltd.); a composition for the lanthionization of hair comprising a sugar (U.S. Pat. Nos. 5,348,737 and 5,641,477, assigned to Avlon Ind. Inc.); the incorporation of xylobiose into cosmetic compositions to provide enhanced moisture retention and reduce excessive roughness and dryness of the skin and hair (U.S. Pat. No. 5,660,838, assigned to Suntory Ltd.); a composition for the regeneration of hair split-ends that contains at least one mono- or di-saccharide (U.S. Pat. No. 4,900,545, assigned to Henkel); hair care compositions to improve hair strength, hold and volume that contain C₅ to C₆ carbohydrates such as glucose; the use of fucose in a hair treatment to prevent split ends (DE29709853, assigned to Goldwell GMBH); and the use of saccharides in a shampoo to improve combing properties and control hair damage (J09059134, assigned to Mikuchi Sangyo K K).

In essence, sugars have been applied to hair for countless reasons from moisturizing to enhancing hair growth (J10279439, assigned to Kureha Chem. Ind. Co. Ltd.). Clearly, however, not all sugars are the same and not all sugars impart the same properties when applied to a keratinous fiber.

The inventors have envisaged the application to at least one keratinous fiber of at least one composition comprising at least one compound comprising at least one C₅ to C₇ saccharide unit substituted with at least one amino group. In particular, the inventors have discovered that compositions and methods using these compositions comprising applying to the at least one keratinous fiber at least one compound comprising at least one C₅ to C₇ saccharide unit substituted with at least one amino group, and optionally at least one film forming agent, and heating the at least one keratinous fiber are useful for durable non-permanent shaping of at least one keratinous fiber or for durable retention of a non-permanent shape of at least one keratinous fiber.

Thus, to achieve at least one of these and other advantages, the present invention, in one aspect, provides a composition for durable non-permanent shaping of at least one keratinous fiber or durable retention of a non-permanent shape of at least one keratinous fiber comprising at least one compound comprising at least one C₅ to C₇ saccharide unit substituted with at least one amino group, and at least one film forming agent different from the at least one compound, wherein the at least one compound and the at least one film forming agent are present in an amount effective to impart a durable non-permanent shape to the at least one keratinous fiber or to durably retain a non-permanent shape of the at least one keratinous fiber. In one embodiment, the composition is heat-activated.

In another embodiment, the present invention is drawn to a method for durable non-permanent shaping of at least one keratinous fiber or durable retention of a non-permanent shape of at least one keratinous fiber comprising applying to the at least one keratinous fiber (i) at least one compound comprising at least one C₅ to C₇ saccharide unit substituted with at least one amino group and (ii) at least one film forming agent different from the at least one compound; and heating the at least one keratinous fiber, wherein the at least one compound and at least one film forming agent are present in an amount effective to impart a durable non-permanent shape to the at least one at least one keratinous fiber or to durably retain a non-permanent shape of the at least one keratinous fiber, and further wherein the composition is applied prior to or during heating.

The present invention, in another aspect, provides a composition for durable non-permanent shaping of at least one keratinous fiber or durable retention of a non-permanent shape of at least one keratinous fiber comprising at least one compound comprising at least one C₅ to C₇ saccharide unit substituted with at least one amino group, wherein the at least one compound is present in an amount effective to impart a durable non-permanent shape to the at least one keratinous fiber or to durably retain a non-permanent shape of the at least one keratinous fiber. In one embodiment, the composition is heat-activated.

In another embodiment, the present invention is drawn to a method for durable non-permanent shaping of at least one keratinous fiber or durable retention of a non-permanent shape of at least one keratinous fiber comprising applying to the at least one keratinous fiber at least one compound comprising at least one C₅ to C₇ saccharide unit substituted with at least one amino group; and heating the at least one keratinous fiber, wherein the at least one compound is present in an amount effective to impart a durable non-permanent shape to the at least one at least one keratinous fiber or to durably retain a non-permanent shape of the at least one keratinous fiber, and further wherein the composition is applied prior to or during heating.

In yet another embodiment, the present invention provides a kit for durable non-permanent shaping of at least one keratinous fiber or durable retention of a non-permanent shape of at least one keratinous fiber comprising at least one compartment, wherein a first compartment comprises a first composition comprising at least one compound comprising at least one C₅ to C₇ saccharide unit substituted with at least one amino group. In one embodiment, at least one compartment comprises at least one additional sugar, different from the at least one compound, and in another embodiment, at least one compartment comprises at least one film forming agent.

Certain terms used herein are defined below:

“At least one” as used herein means one or more and thus includes individual components as well as mixtures/combinations.

“Durable retention of a shape” as used herein means that, following at least six shampoos after treatment, treated hair still retains the ability to retain a particular shape after styling as compared to the ability of untreated hair to retain a particular shape after styling. The ability to retain a shape can be evaluated by measuring, and comparing, the ability to retain a curl under conditions of high relative humidity of the treated hair after six shampoos subsequent to treatment and of the untreated hair after six shampoos in terms of Curl Droop (for example, see Example).

“Durable shaping,” as used herein, refers to holding or keeping a shape of a keratinous fiber until the keratinous fiber is washed with water and/or shampoo. Retention of a shape can be evaluated by measuring, and comparing, the ability to retain a curl under conditions of high relative humidity of the treated hair and of the untreated hair in terms of Curl Droop.

“Heating” refers to the use of elevated temperature (i.e., above 100° C.). In one embodiment, the heating in the inventive method may be provided by directly contacting the at least one keratinous fiber with a heat source, e.g., by heat styling of the at least one keratinous fiber. Non-limiting examples of heat styling by direct contact with the at least one keratinous fiber include flat ironing, and curling methods using elevated temperatures (such as, for example, setting hair in curlers and heating, and curling with a curling iron and/or hot rollers). In another embodiment, the heating in the inventive method may be provided by heating the at least one keratinous fiber with a heat source which may not directly contact the at least one keratinous fiber. Non-limiting examples of heat sources which may not directly contact the at least one keratinous fiber include blow dryers, hood dryers, heating caps and steamers.

“A heat-activated” composition, as used herein, refers to a composition which, for example, shapes the at least one keratinous fiber better than the same composition which is not heated during or after application of the composition. Another example includes a composition which retains a shape of at least one keratinous fiber better than the same composition which is not heated during or after application.

“High humidity” as defined herein refers to atmospheric humidity above 40%.

“Keratinous fibers” as defined herein may be human keratinous fibers, and may be chosen from, for example, hair.

“Non-permanent shaping” of keratinous fibers, as used herein, refers to a method of setting keratinous fibers in a particular shape or style which does not comprise breaking and reforming disulfide bonds within a keratinous fiber.

“Non-permanent shape” of keratinous fibers, as used herein, refers a shape or style of keratinous fibers obtained without breaking and reforming disulfide bonds within a keratinous fiber.

“Oligosaccharides” as defined herein refers to compounds generally comprising from two to ten monosaccharide units, which may be identical or different, bonded together.

“Polysaccharides” as defined herein refers to compounds generally comprising greater than ten monosaccharide units, which may be identical or different, bonded together.

“Polymers” as defined herein comprise copolymers (including terpolymers) and homopolymers. Further, the term “polymers” comprises both oligosaccharides and polysaccharides.

It is to be understood that both the foregoing general description and the following detailed description are exemplary and explanatory only and are not restrictive of the invention, as claimed. Reference will now be made in detail to exemplary embodiments of the present invention.

As described above, sugars have been used in hair care compositions and other treatments for their moisture retaining properties. However, it was unexpectedly discovered by the present inventors that, in addition to retaining moisture, a certain class of sugars imparted a durable non-permanent shape or durable retention of a non-permanent shape or style to at least one keratinous fiber. In particular with respect to hair, compounds comprising at least one C₅ to C₇ saccharide unit substituted with at least one amino group were found to impart good curl formation to the at least one keratinous fiber, and to prevent such curls from drooping, for example, due to humidity. Further, these compounds may impart to the at least one keratinous fiber an ability to retain a particular style even after shampooing the at least one keratinous fiber subsequent to treatment with a composition comprising at least one such compound. This is particularly true when the compounds are applied to the hair, and then the hair is heated.

Thus, the invention provides compositions for durable non-permanent shaping of at least one keratinous fiber or durable retention of a non-permanent shape of at least one keratinous fiber comprising (i) at least one compound comprising at least one C₅ to C₇ saccharide unit substituted with at least one amino group and, optionally, (ii) at least one film forming agent, wherein the at least one compound and, optionally, the at least one film forming agent are present in an amount effective either to impart a durable non-permanent shape to the at least one keratinous fiber or to durably retain a non-permanent shape of the at least one keratinous fiber, depending on the embodiment. In one embodiment, the composition is heat-activated. In another embodiment, the composition both imparts a durable non-permanent shape to the at least one keratinous fiber and durably retains a non-permanent shape of the at least one keratinous fiber. The composition may further comprise at least one additional sugar.

The present invention also provides methods for durable non-permanent shaping of at least one keratinous fiber or for durable retention of a non-permanent shape of at least one keratinous fiber comprising applying to the at least one keratinous fiber a composition comprising (i) at least one compound comprising at least one C₅ to C₇ saccharide unit substituted with at least one amino group, and, optionally, (ii) at least one film forming agent; and heating the at least one keratinous fiber. The composition may be applied prior to or during heating. Further, the at least one compound and, optionally, the at least one film forming agent are present in an amount effective either to impart a durable non-permanent shape to the at least one keratinous fiber or to durably retain a non-permanent shape of the at least one keratinous fiber, depending on the embodiment. In one embodiment, the composition both imparts a durable non-permanent shape to the at least one keratinous fiber and durably retains a non-permanent shape of the at least one keratinous fiber. The composition may further comprise at least one additional sugar.

According to certain embodiments of the present invention, the at least one compound may be used in conjunction with at least one film-forming agent, such as, for example, film forming polymers and resins. For example, the film forming polymers may be chosen from cationic polymers, anionic polymers and nonionic polymers. Non-limiting examples of the at least one film forming agent are those listed at pages 1744 to 1747 of the CTFA International Cosmetic Ingredient Dictionary, 8^{th} edition (2000). In one embodiment, the at least one film forming agent may be chosen from water soluble compounds, oil soluble compounds and compounds soluble in organic solvents. According to the present invention, the at least one film forming agent may be present in an amount generally ranging from 0.01% to 30% of active material by weight relative to the total weight of the composition, such as from 0.1% to 10% of active material by weight. One of ordinary skill in the art will recognize that the at least one film forming agent according to the present invention may be commercially available, and may come from suppliers in the form of a dilute solution. The amounts of the at least one film forming agent disclosed herein therefore reflect the weight percent of active material.

Non-limiting examples of the at least one film forming agent are those disclosed in WO 01/18096, the disclosure of which is incorporated herein by reference. Other non-limiting examples of the at least one film forming agent include copolymers derived from (i) at least one vinyl monomer comprising at least one quaternary ammonium group and (ii) at least one additional monomer chosen from acrylamide, methacrylamide, alkyl acrylamides, dialkyl acrylamides, alkyl methacrylamides, dialkyl methacrylamides, alkyl acrylate, alkyl methacrylate, vinyl caprolactone, vinyl pyrrolidone, vinyl esters, vinyl alcohol, maleic anhydride, propylene glycol, and ethylene glycol.

Further non-limiting examples of the at least one film forming agent include:

vinyl acetate/vinyl tert butylbenzoate/crotonic acid terpolymers such as those described in U.S. Pat. No. 4,282,203, the disclosure of which is incorporated herein by reference;

N-octylacrylamide/methyl methacrylate/hydroxypropyl methacrylate/acrylic acid/tert-butylamino-ethyl methacrylate copolymers such as those sold by NATIONAL STARCH under the name “AMPHOMER LV-71”;

corn starch/polyvinylpyrrolidone copolymers such as Corn Starch Modified sold by National Starch and Chemicals under the name Amaize®;

vinylpyrrolidone/vinyl acetate copolymers such as those sold by BASF under the name “LUVISKOL VA 64 Powder”;

vinyl acetate/crotonic acid/vinyl neodecanoate terpolymers such as those sold by NATIONAL STARCH under the name “RESYN® 28-2930”;

acrylic acid/ethyl acrylate/N-tert-butylacrylamide terpolymers such as those sold by BASF under the name “ULTRA-HOLD 8”;

acrylic acid/acrylates/hydroxyacrylates/succinic acid copolymers such as Acrylates/C1-2 succinates hydroxyacrylates copolymer sold by ISP as ALLIANZ LT-120;

vinyl acetate/crotonic acid (90/10) copolymers such as those sold by BASF under the name “LUVISET CA 66”;

acrylic acid/methacrylic acid/acrylates/methacrylates copolymers such as Acrylates Copolymer sold by Amerchol Corp. (Edison, N.J., USA); and

vinylcaprolactam/vinylpyrrolidone/dimethylamino ethyl methacrylate copolymers such as those sold by GAF under the name “POLYMER ACP-1018”.

Further non-limiting examples of the at least one film forming agent include:

copolymers derived from (i) 1-vinyl-2-pyrrolidone and (ii) 1-vinyl-3-methylimidazolium salt (CTFA designation: polyquaternium-16), which is commercially available from BASF Corporation under the LUVIQUAT tradename (e.g., LUVIQUAT FC 370);

copolymers derived from reaction of (i) vinylcaprolactam and (ii) vinylpyrroldone with methylvinylimidazolium methosulfate, (CTFA designation: polyquaternium-46), which is commercially available from BASF;

copolymers derived from (i) vinylpyrrolidone and (ii) quaternized imidazoline monomers (CTFA designation: polyquaternium-44), which is commercially available from BASF;

copolymers derived from (i) 1-vinyl-2-pyrrolidone and (ii) 1-vinyl-3-methylimidazolium salt (CTFA designation: polyquaternium-16), which is commercially available from BASF Corporation under the LUVIQUAT tradename (e.g., LUVIQUAT FC 370);

poly(vinylamine), optionally quaternized;

poly-4-vinyl pyridine, optionally quaternized;

poly(ethyleneimine), optionally quaternized;

dimethyldiallylammonium chloride homopolymer (CTFA designation: polyquaternium-6);

copolymers derived from (i) acrylamide and (ii) dimethyidiallylammonium chloride (CTFA designation: polyquaternium-7);

copolymers derived from (i) dimethyidiallylammonium chloride and (ii) sodium acrylate (CTFA designation: Polyquaternium-22); and

terpolymers derived from (i) dimethyldiallylammonium chloride, (ii) acrylic amide and (iii) sodium acrylate (CTFA designation: Polyquaternium-39).

Other non-limiting examples of the at least one film forming agent include derivatives of polysaccharide polymers such as cationic cellulose derivatives, for example, cationic cellulose, which is available from Amerchol Corp. (Edison, N.J., USA) in their Polymer JR™, LR™ and SR™ series of polymers, as salts of hydroxyethyl cellulose reacted with trimethyl ammonium substituted epoxide (CTFA designation: polyquaternium-10); polymeric quaternary ammonium salts of hydroxyethyl cellulose reacted with lauryl dimethyl ammonium-substituted epoxide (CTFA designation: polyquaternium-24), which is available from Amerchol Corp. (Edison, N.J., USA) under the tradename Polymer LM-200™; and cationic starch and derivatives thereof, such as quaternary starch, which is available from Croda.

In one embodiment, the at least one film forming agent is chosen from cationic polymers such as polyquaternium-16, polyquaternium-46, and polyquaternium-44. In another embodiment, the at least one film forming agent is chosen from nonionic polymers such as polymers derived from (1) corn starch and (2) polyvinylpyrrolidone; and copolymers derived from (1) vinyl acetate and (2) vinylpyrrolidone. In yet another embodiment, the at least one film forming agent is chosen from anionic polymers such as polymers derived from (1) vinyl acetate, (2) crotonic acid and (3) vinyl neodecanoate, polymers derived from (1) acrylic acid, (2) acrylates, (3) hydroxyacrylates and (4) succinic acid, and polymers derived from at least two monomers chosen from acrylic acid, methacrylic acid, esters of acrylic acid and esters of methacrylic acid. The at least one film forming agent chosen from anionic polymers can be neutralized in order to render the anionic polymers soluble.

The at least one C₅ to C₇ saccharide unit according to the present invention may be chosen from any pentose, hexose and heptose. Further, the at least one C₅ to C₇ saccharide unit can be chosen from the D-form, L-form and mixtures of any of the foregoing Non-limiting examples of C₅ to C₇ saccharide units are aldopentoses (such as xylose, arabinose, lyxose, and ribose), ketopentoses (such as ribulose and xylulose), aldohexoses (such as glucose and galactose), ketohexoses (such as fructose and sorbose), and heptoses (such as aldoheptoses and ketoheptoses, e.g., galactoheptulose and glucoheptulose). The at least one C₅ to C₇ saccharide unit may be chosen from those comprising aldehyde groups (aldoses), furanoses and other ring structures. The at least one C₅ to C₇ saccharide unit may be further substituted with at least one group different from the at least one amino group.

Derivatives of C₅ to C₇ saccharide units may also be used as the at least one C₅ to C₇ saccharide unit in the present invention. For example, ammonias or primary amines may react with the aldehyde or ketone group of a saccharide unit to form an imine derivative (i.e., a compound containing the functional group C═N). These imine compounds are sometimes also referred to as Schiff bases. Other non-limiting examples of derivatives of C₅ to C₇ saccharide units are hemiacetal derivatives of C₅ to C₇ saccharide units, hemiketal derivatives of C₅ to C₇ saccharide units and any oxidized derivatives of C₅ to C₇ saccharide units. These derivatives may be formed, for example, from the reaction of the aldehyde or ketone group of a saccharide unit with an alcohol. As previously mentioned, the at least one C₅ to C₇ saccharide unit may be further substituted with at least one group different from the at least one amino group. Thus, in one embodiment, the derivatives of C₅ to C₇ saccharide units may be further substituted with at least one group different from the at least one amino group.

According to the present invention, the at least one amino group may be chosen from substituted and unsubstituted amino groups. For example, the at least one amino group may be chosen from N-acetyl amino groups.

Further, the at least one C₅ to C₇ saccharide unit may be substituted with the at least one amino group at any position on the saccharide unit. For example, in one embodiment, the at least one C₅ to C₇ saccharide unit is substituted with the at least one amino group at the C1 position of the at least one C₅ to C₇ saccharide unit. In another embodiment, the at least one C₅ to C₇ saccharide unit is substituted with the at least one amino group at the C2 position of the at least one C₅ to C₇ saccharide unit.

Non-limiting examples of the at least one compound include C₅ monosaccharides substituted with at least one amino group, C₆ monosaccharides substituted with at least one amino group, C₇ monosaccharides substituted with at least one amino group, polymers comprising at least one C₅ monosaccharide substituted with at least one amino group, polymers comprising at least one C₆ monosaccharide substituted with at least one amino group, polymers comprising at least one C₇ monosaccharide substituted with at least one amino group, and glycoproteins comprising at least one C₅ to C₇ saccharide unit substituted with at least one amino group. In one embodiment, the at least one compound is chosen from oligosaccharides derived from the at least one C₅ to C₇ saccharide unit substituted with at least one amino group which may be further substituted with at least one group different from the at least one amino group.

Non-limiting examples of C₅ monosaccharides substituted with at least one amino group are pentosamines. In one embodiment, the pentosamines are chosen from aldopentosamines and ketopentosamines (such as xylosamine, arabinosamine, lyxosamine, ribosamine, ribulosamine and xylulosamine).

Non-limiting examples of C₆ monosaccharides substituted with at least one amino group include hexosamines (such as aldohexosamines and ketohexosamines). In one embodiment, for example, the hexosamines are chosen from glucosamine, galactosamine, allosamine, altrosamine, mannosamine, gulosamine, idosamine, galactosamine, and talosamine. In another embodiment, the at least one compound is glucosamine, and in another embodiment, is galactosamine.

Non-limiting examples of C₇ monosaccharides substituted with at least one amino group are heptosamines. For example, heptosamines may be chosen from aldoheptosamines and ketoheptosamines.

According to the present invention, the at least one compound comprising at least one C₅ to C₇ saccharide unit substituted with at least one amino group is present in the composition in an amount generally ranging from 0.01% to 10% by weight relative to the total weight of the composition, and in another embodiment from 0.1% to 5% by weight.

The compositions of the present invention and those used in the methods of the present invention may further comprise at least one additional sugar which is different from the at least one compound comprising at least one C₅ to C₇ saccharide unit substituted with at least one amino group. The at least one additional sugar may, for example, aid in moisture retention. The effectiveness of a sugar in aiding in moisture retention may be measured by monitoring a DSC peak at a temperature ranging from 75° C. to 200° C.

The at least one additional sugar may be chosen from any sugar, carbohydrate and carbohydrate moiety. Non-limiting examples of the at least one additional sugar are monosaccharides, which include, but are not limited to, three to seven carbon sugars such as pentoses (for example, ribose, arabinose, xylose, lyxose, ribulose, and xylulose) and hexoses (for example, allose, altrose, glucose, mannose, gulose, idose, galactose, talose, sorbose, psicose, fructose, and tagatose); oligosaccharides such as disaccharides (such as maltose, sucrose, cellobiose, trehalose and lactose); and polysaccharides such as starch, dextrins, cellulose and glycogen. In one embodiment, the at least one additional sugar of the invention is chosen from any aldoses and ketoses. Further, the at least one additional sugar may be substituted or unsubstituted.

According to the present invention, the at least one additional sugar is present in the composition in an amount generally ranging from 0.01% to 10% by weight relative to the total weight of the composition, such as from 0.1 % to 5% by weight.

The compositions of the present invention and those used in the methods of the present invention may be in the form of a liquid, an oil, a paste, a stick, a dispersion, an emulsion, a lotion, a gel, or a cream. Further, these compositions may further comprise at least one suitable additive chosen from additives commonly used in compositions for keratinous fibers. Non-limiting examples of the at least one suitable additive include anionic surfactants, cationic surfactants, nonionic surfactants, amphoteric surfactants, fragrances, penetrating agents, antioxidants, sequestering agents, opacifying agents, solubilizing agents, emollients, colorants, screening agents (such as sunscreens and UV filters), preserving agents, proteins, vitamins, silicones, polymers such as thickening polymers, plant oils, mineral oils, synthetic oils and any other additive conventionally used in compositions for the care and/or treatment of keratinous fibers.

Needless to say, a person skilled in the art will take care to select the at least one suitable additive such that the advantageous properties of the composition in accordance with the invention are not, or are not substantially, adversely affected by the addition(s) envisaged.

The compositions of the present invention and those used in the methods of the present invention may also be provided as one-part compositions comprising at least one compound comprising at least one C₅ to C₇ saccharide unit substituted with at least one amino group and, optionally, at least one additional sugar, and further, optionally at least one film forming agent, or in the form of a multi-component treatment or kit. The skilled artisan, based on the stability of the composition and the application envisaged, will be able to determine how the composition and/or multicomponent compositions should be stored and mixed. For example, simple sugars such as C₅ to C₇ monosaccharides are known to be stable at pH levels ranging from 4 to 9. In compositions where the pH range is below or above these levels, the sugars would be stored separately and added to the composition only at the time of application.

Thus, the present invention also relates to a kit for durable non-permanent shaping of at least one keratinous fiber or for durable retention of a non-permanent shape of at least one keratinous fiber comprising at least one compartment, wherein a first compartment comprises a first composition comprising at least one compound comprising at least one C₅ to C₇ saccharide unit substituted with at least one amino group. In one embodiment, the first composition further comprises at least one additional sugar, different from the at least one compound, while in another embodiment, the first composition further comprises at least one film forming agent. In yet another embodiment, the kit further comprises a second compartment comprising a composition comprising at least one film forming agent.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. The following example is intended to illustrate the invention without limiting the scope as a result.

### EXAMPLE

The following procedure was used to treat the hair and measure the Curl Droop: Hair swatches (2 g., 6.5-7.5 in.) were treated with a solution containing 6% Corn Starch Modified and 1% Glucosamine (0.5 g solution/g of hair) then blow dried. The hair swatches were then heated with a flat iron for 1 minute and then shampooed with 10% sodium laureth sulfate (SLES). The treatment was repeated up to 8 times, as indicated. The treated hair swatches were shampooed 2, 4, and 6 times, then styled with a curling iron for 30 seconds and placed in a humidity chamber at 90% relative humidity to measure the Curl Droop. As the curl slowly relaxed in the humidity chamber, the length of the hair swatches was measured every minute (up to 15 minutes).

The Curl Droop was calculated as:

[(Lₒ−Lₜ)/(Lₒ−Lᵢ)]×100

Where:

Lₒ represents the original length of fully extended hair

Lₜ represents the length of the hair at time t in the humidity chamber

Lᵢ represents the initial length of the hair at time 0 in the humidity chamber (i.e., after styling with a curling iron for 30 seconds)

A higher Curl Droop represents a better curl retention.

TABLE 1
Curl Droop of Hair Treated with Corn Starch Modified

| Solution Comprising 6% (active) | O | 5 | 10 | 15 |
|---|---|---|---|---|
| Corn Starch Modified | minutes | minutes | minutes | minutes |
| Without glucosamine, after | 100 | 85 | 75 | 62 |
| treatment | | | | |
| With glucosamine, after treatment | 100 | 88 | 76 | 65 |
| Without glucosamine, after 2 | 100 | 83 | 68 | 37 |
| shampoos | | | | |
| With glucosamine, after 2 | 100 | 85 | 72 | 40 |
| shampoos | | | | |
| Without glucosamine, after 4 | 100 | 81 | 62 | 27 |
| shampoos | | | | |
| With glucosamine, after 4 | 100 | 83 | 71 | 32 |
| shampoos | | | | |
| Without glucosamine, after 6 | 100 | 77 | 47 | 13 |
| shampoos | | | | |
| With glucosamine, after 6 | 100 | 80 | 66 | 19 |
| shampoos | | | | |

The data showed that hair treated with at least one film forming agent (Corn Starch Modified), at least one compound comprising at least one C₅ to C₇ saccharide unit substituted with at least one amino group (glucosamine) and heat had a higher curl retention than hair treated with at least one film forming agent (Corn Starch Modified) and heat but without at least one compound comprising at least one C₅ to C₇ saccharide unit substituted with at least one amino group even after 6 shampoos.

What is claimed is:

1. A composition for durable non-permanent shaping of least one keratinous fiber or durable retention of a non-permanent shape of least one keratinous fiber comprising:
(a) at least one compound comprising at least one C₅ to C₇ saccharide unit substituted with at least one amino group; and
(b) at least one film forming agent,
wherein said at least one compound and said at least one film forming agent are present in an amount effective to impart a durable non-permanent shape to said at least one keratinous fiber or to durably retain a non-permanent shape of said at least one keratinous fiber.

2. A composition according to claim 1, wherein said at least one film forming agent is chosen from cationic polymers.

3. A composition according to claim 2, wherein said cationic polymers are chosen from polyquaternium-16, polyquaternium-46 and polyquaternium-44.

4. A composition according to claim 1, wherein said at least one film forming agent is chosen from nonionic polymers.

5. A composition according to claim 4, wherein said nonionic polymers are chosen from:
(i) polymers derived from (1) corn starch and (2) polyvinylpyrrolidone; and
(ii) copolymers derived from (1) vinyl acetate and (2) vinylpyrrolidone.

6. A composition according to claim 1, wherein said at least one film forming agent is chosen from anionic polymers.

7. A composition according to claim 6, wherein said anionic polymers are chosen from:
(i) polymers derived from (1) vinyl acetate, (2) crotonic acid and (3) vinyl neodecanoate;
(ii) polymers derived from (1) acrylic acid, (2) acrylates, (3) hydroxyacrylates and (4) succinic acid; and
(iii) polymers derived from at least two different monomers each chosen from acrylic acid, methacrylic acid, esters of acrylic acid, and esters of methacrylic acid.

8. A composition according to claim 6, wherein said anionic polymers are neutralized.

9. A composition according to claim 1, wherein said at least one film forming agent is present in said composition in an amount ranging from 0.01% to 30% by weight relative to the total weight of the composition.

10. A composition according to claim 9, wherein said at least one film forming agent is present in said composition in an amount ranging from 0.1% to 10% by weight relative to the total weight of the composition.

11. A composition according to claim 1, wherein said at least one amino group is chosen from unsubstituted amino groups and substituted amino groups.

12. A composition according to claim 1, wherein said at least one C₅ to C₇ saccharide unit is further substituted with at least one group different from said at least one amino group.

13. A composition according to claim 1, wherein said at least one C₅ to C₇ saccharide unit is substituted with said at least one amino group at C1 of said saccharide unit.

14. A composition according to claim 1, wherein said at least one C₅ to C₇ saccharide unit is substituted with said at least one amino group at C2 of said saccharide unit.

15. A composition to claim 1, wherein said at least one compound is chosen from C₅ monosaccharides substituted with at least one amino group, C₆ monosaccharides substituted with at least one amino group, C₇ monosaccharides substituted with at least one amino group, polymers comprising at least one C₅ monosaccharide substituted with at least one amino group, polymers comprising at least one C₆ monosaccharide substituted with at least one amino group, polymers comprising at least one C₇ monosaccharide substituted with at least one amino group, and glycoproteins comprising at least one C₅ to C₇ saccharide unit substituted with at least one amino group.

16. A composition according to claim 15, wherein said C₅ monosaccharides substituted with at least one amino group are chosen from pentosamines.

17. A composition according to claim 16, wherein said pentosamines are chosen from aldopentosamines and ketopentosamines.

18. A composition according to claim 17, wherein said pentosamines are chosen from xylosamine, arabinosamine, lyxosamine, ribosamine, ribulosamine and xylulosamine.

19. A composition according to claim 15, wherein said C₆ monosaccharides substituted with at least one amino group are chosen from hexosamines.

20. A composition according to claim 19, wherein said hexosamines are chosen from aldohexosamines and ketohexosamines.

21. A composition according to claim 20, wherein hexosamines are chosen from glucosamine, galactosamine, allosamine, altrosamine, mannosamine, gulosamine, idosamine, galactosamine, and talosamine.

22. A composition according to claim 15, wherein said C₇ monosaccharides substituted with at least one amino group are chosen from heptosamines.

23. A composition according to claim 22, wherein said heptosamines are chosen from aldoheptosamines and ketoheptosamines.

24. A composition according to claim 1, wherein said at least one compound is chosen from oligosaccharides derived from said at least one C₅ to C₇ saccharide unit substituted with at least one amino group.

25. A composition according to claim 1, wherein said at least one C₅ to C₇ saccharide unit is chosen from furanoses and derivatives thereof.

26. A composition according to claim 1, wherein said at least one C₅ to C₇ saccharide unit is chosen from derivatives of C₅ to C₇ saccharide units.

27. A composition according to claim 26, wherein said derivatives of C₅ to C₇ saccharide units are chosen from imine derivatives of C₅ to C₇ saccharide units, hemiacetal derivatives of C₅ to C₇ saccharide units, hemiketal derivatives of C₅ to C₇ saccharide units, and oxidized derivatives of C₅ to C₇ saccharide units.

28. A composition according to claim 26, wherein said derivatives of C₅ to C₇ saccharide units are further substituted with at least one group different from said at least one amino group.

29. A composition according to claim 1, wherein said at least one compound is chosen from lyxosylamine.

30. A composition according to claim 1, wherein said at least one compound is chosen from glucosamine.

31. A composition according to claim 1, wherein said at least one compound is chosen from galactosamine.

32. A composition according to claim 1, wherein said at least one compound is present in said composition in an amount ranging from 0.01% to 10% by weight relative to the total weight of the composition.

33. A composition according to claim 32, wherein said at least one compound is present in said composition in an amount ranging from 0.1% to 5% by weight relative to the total weight of the composition.

34. A composition according to claim 1, wherein said composition further comprises at least one additional sugar, said at least one additional sugar being different from said at least one compound comprising at least one C₅ to C₇ saccharide unit substituted with at least one amino group.

35. A composition according to claim 34, wherein said at least one additional sugar is chosen from monosaccharides, oligosaccharides and polysaccharides.

36. A composition according to claim 35, wherein said monosaccharides are chosen from hexoses.

37. A composition according to claim 36, wherein said hexoses are chosen from allose, altrose, glucose, mannose, gulose, idose, galactose, talose, sorbose, psicose, fructose, and tagatose.

38. A composition according to claim 34, wherein said at least one additional sugar is present in said composition in an amount ranging from 0.01% to 10% by weight relative to the total weight of the composition.

39. A composition according to claim 38, wherein said at least one additional sugar is present in said composition in an amount ranging from 0.1% to 5% by weight relative to the total weight of the composition.

40. A composition according to claim 1, wherein said composition is in the form of a liquid, oil, paste, stick, dispersion, emulsion, lotion, gel, or cream.

41. A composition according to claim 1, wherein said at least one keratinous fiber is hair.

42. A composition according to claim 1, further comprising at least one suitable additive chosen from anionic surfactants, cationic surfactants, nonionic surfactants, amphoteric surfactants, fragrances, penetrating agents, antioxidants, sequestering agents, opacifying agents, solubilizing agents, emollients, colorants, screening agents, preserving agents, proteins, vitamins, silicones, polymers, plant oils, mineral oils, and synthetic oils.

43. A composition according to claim 1, wherein said composition is heat-activated.

44. A method for durable non-permanent shaping of at least one keratinous fiber or for durable retention of a non-permanent shape of at least one keratinous fiber comprising:
applying to said at least one keratinous fiber a composition comprising:
(a) at least one compound comprising at least one C₅ to C₇ saccharide unit substituted with at least one amino group, and
(b) at least one film forming agent; and
heating said at least one keratinous fiber;
wherein said at least one compound and said at least one film forming agent are present in an amount effective to impart a durable non-permanent shape to said at least one keratinous fiber or to durably retain a non-permanent shape of said at least one keratinous fiber, and
further wherein said composition is applied prior to said heating or during said heating.

45. A method according to claim 44, further comprising wetting said at least one keratinous fiber with water prior to said application.

46. A method according to claim 44, further comprising shampooing said at least one keratinous fiber subsequent to said heating.

47. A method according to claim 46, further comprising rinsing said at least one keratinous fiber subsequent to said shampooing.

48. A method according to claim 44, wherein said composition is applied prior to and during said heating.

49. A method according to claim 44, wherein said at least one film forming agent is chosen from film forming polymers and film forming resins.

50. A method according to claim 49, wherein said film forming polymers are chosen from cationic polymers.

51. A method according to claim 50, wherein said cationic polymers are chosen from polyquaternium-16, polyquaternium-46 and polyquaternium-44.

52. A method according to claim 49, wherein film forming polymers are chosen from nonionic polymers.

53. A method according to claim 52, wherein said nonionic polymers are chosen from:
(i) polymers derived from (1) corn starch and (2) polyvinylpyrrolidone; and
(ii) copolymers derived from (1) vinyl acetate and (2) vinylpyrrolidone.

54. A method according to claim 49, wherein said film forming polymers are chosen from anionic polymers.

55. A method according to claim 54, wherein said anionic polymers are chosen from:
(i) polymers derived from (1) vinyl acetate, (2) crotonic acid and (3) vinyl neodecanoate;
(ii) polymers derived from (1) acrylic acid, (2) acrylates, (3) hydroxyacrylates and (4) succinic acid; and
(iii) polymers derived from at least two different monomers each chosen from acrylic acid, methacrylic acid, esters of acrylic acid and esters of methacrylic acid.

56. A method according to claim 54, wherein said anionic polymers are neutralized.

57. A method according to claim 44, wherein said at least one film forming agent is present in said composition in an amount ranging from 0.01% to 30% by weight relative to the total weight of the composition.

58. A method according to claim 57, wherein said at least one film forming agent is present in said composition in an amount ranging from 0.1% to 10% by weight relative to the total weight of the composition.

59. A method according to claim 44, wherein said at least one amino group is chosen from unsubstituted amino groups and substituted amino groups.

60. A method according to claim 44, wherein said at least one C₅ to C₇ saccharide unit is further substituted with at least one group different from said at least one amino group.

61. A method according to claim 44, wherein said at least one C₅ to C₇ saccharide unit is substituted with said at least one amino group at C1 of said saccharide unit.

62. A method according to claim 44, wherein said at least one C₅ to C₇ saccharide unit is substituted with said at least one amino group at C2 of said saccharide unit.

63. A method according to claim 44, wherein said at least one compound is chosen from C₅ monosaccharides substituted with at least one amino group, C₆ monosaccharides substituted with at least one amino group, C₇ monosaccharides substituted with at least one amino group, polymers comprising at least one C₅ monosaccharide substituted with at least one amino group, polymers comprising at least one C₆ monosaccharide substituted with at least one amino group, polymers comprising at least one C₇ monosaccharide substituted with at least one amino group, and glycoproteins comprising at least one C₅ to C₇ saccharide unit substituted with at least one amino group.

64. A method according to claim 63, wherein said C₅ monosaccharides substituted with at least one amino group are chosen from pentosamines.

65. A method according to claim 64, wherein said pentosamines are chosen from aldopentosamines and ketopentosamines.

66. A method according to claim 65, wherein said pentosamines are chosen from xylosamine, arabinosamine, lyxosamine, ribosamine, ribulosamine and xylulosamine.

67. A method according to claim 63, wherein said C₆ monosaccharides substituted with at least one amino group are chosen from hexosamines.

68. A method according to claim 67, wherein said hexosamines are chosen from aldohexosamines and ketohexosamines.

69. A method according to claim 68, wherein hexosamines are chosen from glucosamine, galactosamine, allosamine, altrosamine, mannosamine, gulosamine, idosamine, galactosamine, and talosamine.

70. A method according to claim 63, wherein said C₇ monosaccharides substituted with at least one amino group are chosen from heptosamines.

71. A method according to claim 70, wherein said heptosamines are chosen from aldoheptosamines and ketoheptosamines.

72. A method according to claim 44, wherein said at least one compound is chosen from oligosaccharides derived from said at least one C₅ to C₇ saccharide unit substituted with at least one amino group.

73. A method according to claim 44, wherein said at least one C₅ to C₇ saccharide unit is chosen from furanoses and derivatives thereof.

74. A method according to claim 44, wherein said at least one C₅ to C₇ saccharide unit is chosen from derivatives of C₅ to C₇ saccharide units.

75. A method according to claim 74, wherein said derivatives of C₅ to C₇ saccharide units are chosen from imine derivatives of C₅ to C₇ saccharide units, hemiacetal derivatives of C₅ to C₇ saccharide units, hemiketal derivatives of C₅ to C₇ saccharide units, and oxidized derivatives of C₅ to C₇ saccharide units.

76. A method according to claim 74, wherein said derivatives of C₅ to C₇ saccharide units are further substituted with at least one group different from said at least one amino group.

77. A method according to claim 44, wherein said at least one compound is chosen from lyxosylamine.

78. A method according to claim 44, wherein said at least one compound is chosen from glucosamine.

79. A method according to claim 44, wherein said at least one compound is chosen from galactosamine.

80. A method according to claim 44, wherein said at least one compound is present in said composition in an amount ranging from 0.01% to 10% by weight relative to the total weight of the composition.

81. A method according to claim 80, wherein said at least one compound is present in said composition in an amount ranging from 0.1% to 5% by weight relative to the total weight of the composition.

82. A method according to claim 44, wherein said composition further comprises at least one additional sugar, said at least one additional sugar being different from said at least one compound comprising at least one C₅ to C₇ saccharide unit substituted with at least one amino group and derivatives thereof.

83. A method according to claim 82, wherein said at least one additional sugar is chosen from monosaccharides, oligosaccharides and polysaccharides.

84. A method according to claim 83, wherein said monosaccharides are chosen from hexoses.

85. A method according to claim 84, wherein said hexoses are chosen from allose, altrose, glucose, mannose, gulose, idose, galactose, talose, sorbose, psicose, fructose, and tagatose.

86. A method according to claim 82, wherein said at least one additional sugar is present in said composition in an amount ranging from 0.01 % to 10% by weight relative to the total weight of the composition.

87. A method according to claim 86, wherein said at least one additional sugar is present in said composition in an amount ranging from 0.1% to 5% by weight relative to the total weight of the composition.

88. A method according to claim 44, wherein said composition is in the form of a liquid, oil, paste, stick, dispersion, emulsion, lotion, gel, or cream.

89. A method according to claim 44, wherein said at least one keratinous fiber is hair.

90. A method according to claim 44, wherein said composition further comprises at least one suitable additive chosen from anionic surfactants, cationic surfactants, nonionic surfactants, amphoteric surfactants, fragrances, penetrating agents, antioxidants, sequestering agents, opacifying agents, solubilizing agents, emollients, colorants, screening agents, preserving agents, proteins, vitamins, silicones, polymers, plant oils, mineral oils, and synthetic oils.

91. A method according to claim 44, wherein said composition imparts a durable non-permanent shape to said at least one keratinous fiber and durably retains a non-permanent shape of said at least one keratinous fiber.

92. A method for durable non-permanent shaping of at least one keratinous fiber or for durable retention of a non-permanent shape of at least one keratinous fiber comprising:
applying to said at least one keratinous fiber a composition comprising at least one compound comprising at least one C₅ to C₇ saccharide unit substituted with at least one amino group; and
heating said at least one keratinous fiber;
wherein said at least one compound is present in an amount effective to impart a durable non-permanent shape to said at least one keratinous fiber or to durably retain a non-permanent shape of said at least one keratinous fiber, and
further wherein said composition is applied prior to said heating or during said heating.

93. A method according to claim 92, further comprising wetting said at least one keratinous fiber with water prior to said application.

94. A method according to claim 92, further comprising shampooing said at least one keratinous fiber subsequent to said heating.

95. A method according to claim 94, further comprising rinsing said at least one keratinous fiber subsequent to said shampooing.

96. A method according to claim 92, wherein said composition is applied prior to and during said heating.

97. A method according to claim 92, wherein said at least one amino group is chosen from unsubstituted amino groups and substituted amino groups.

98. A method according to claim 92, wherein said at least one C₅ to C₇ saccharide unit is further substituted with at least one group different from said at least one amino group.

99. A method according to claim 92, wherein said at least one C₅ to C₇ saccharide unit is substituted with said at least one amino group at C1 of said saccharide unit.

100. A method according to claim 92, wherein said at least one C₅ to C₇ saccharide unit is substituted with said at least one amino group at C2 of said saccharide unit.

101. A method according to claim 92, wherein said at least one compound is chosen from C monosaccharides substituted with at least one amino group, C₆ monosaccharides substituted with at least one amino group, C₇ monosaccharides substituted with at least one amino group, polymers comprising at least one C₅ monosaccharide substituted with at least one amino group, polymers comprising at least one C₆ monosaccharide substituted with at least one amino group, polymers comprising at least one C₇ monosaccharide substituted with at least one amino group, and glycoproteins comprising at least one C₅ to C₇ saccharide unit substituted with at least one amino group.

102. A method according to claim 101, wherein said C₅ monosaccharides substituted with at least one amino group are chosen from pentosamines.

103. A method according to claim 102, wherein said pentosamines are chosen from aldopentosamines and ketopentosamines.

104. A method according to claim 103, wherein said pentosamines are chosen from xylosamine, arabinosamine, lyxosamine, ribosamine, ribulosamine and xylulosamine.

105. A method according to claim 101, wherein said C₆ monosaccharides substituted with at least one amino group are chosen from hexosamines.

106. A method according to claim 105, wherein said hexosamines are chosen from aldohexosamines and ketohexosamines.

107. A method according to claim 106, wherein said hexosamines are chosen from glucosamine, galactosamine, allosamine, altrosamine, mannosamine, gulosamine, idosamine, galactosamine, and talosamine.

108. A method according to claim 101, wherein said C₇ monosaccharides substituted with at least one amino group are chosen from heptosamines.

109. A method according to claim 108, wherein said heptosamines are chosen from aldoheptosamines and ketoheptosamines.

110. A method according to claim 92, wherein said at least one compound is chosen from oligosaccharides derived from said at least one C₅ to C₇ saccharide unit substituted with at least one amino group.

111. A method according to claim 92, wherein said at least one C₅ to C₇ saccharide unit is chosen from furanoses and derivatives thereof.

112. A method according to claim 92, wherein said at least one C₅ to C₇ saccharide unit is chosen from derivatives of C₅ to C₇ saccharide units.

113. A method according to claim 112, wherein said derivatives of C₅ to C₇ saccharide units are chosen from imine derivatives of C₅ to C₇ saccharide units, hemiacetal derivatives of C₅ to C₇ saccharide units, hemiketal derivatives of C₅ to C₇ saccharide units, and oxidized derivatives of C₅ to C₇ saccharide units.

114. A method according to claim 112, wherein said derivatives of C₅ to C₇ saccharide units are further substituted with at least one group different from said at least one amino group.

115. A method according to claim 92, wherein said at least one compound is chosen from lyxosylamine.

116. A method according to claim 92, wherein said at least one compound is chosen from glucosamine.

117. A method according to claim 92, wherein said at least one compound is chosen from galactosamine.

118. A method according to claim 92, wherein said at least one compound is present in said composition in an amount ranging from 0.01% to 10% by weight relative to the total weight of the composition.

119. A method according to claim 118, wherein said at least one compound is present in said composition in an amount ranging from 0.1% to 5% by weight relative to the total weight of the composition.

120. A method according to claim 92, wherein said composition further comprises at least one additional sugar, said at least one additional sugar being different from said at least one compound comprising at least one C₅ to C₇ saccharide unit substituted with at least one amino group and derivatives thereof.

121. A method according to claim 120, wherein said at least one additional sugar is chosen from monosaccharides, oligosaccharides and polysaccharides.

122. A method according to claim 121, wherein said monosaccharides are chosen from hexoses.

123. A method according to claim 122, wherein said hexoses are chosen from allose, altrose, glucose, mannose, gulose, idose, galactose, talose, sorbose, psicose, fructose, and tagatose.

124. A method according to claim 120, wherein said at least one additional sugar is present in said composition in an amount ranging from 0.01% to 10% by weight relative to the total weight of the composition.

125. A method according to claim 124, wherein said at least one additional sugar is present in said composition in an amount ranging from 0.1% to 5% by weight relative to the total weight of the composition.

126. A method according to claim 92, wherein said composition is in the form of a liquid, oil, paste, stick, dispersion, emulsion, lotion, gel, or cream.

127. A method according to claim 92, wherein said at least one keratinous fiber is hair.

128. A method according to claim 92, wherein said composition further comprises at least one suitable additive chosen from anionic surfactants, cationic surfactants, nonionic surfactants, amphoteric surfactants, fragrances, penetrating agents, antioxidants, sequestering agents, opacifying agents, solubilizing agents, emollients, colorants, screening agents, preserving agents, proteins, vitamins, silicones, polymers, plant oils, mineral oils, and synthetic oils.

129. A method according to claim 92, wherein said composition imparts a durable non-permanent shape to said at least one keratinous fiber and durably retains a non-permanent shape of said at least one keratinous fiber.

130. A composition for durable non-permanent shaping of least one keratinous fiber or durable retention of a non-permanent shape of least one keratinous fiber comprising at least one compound comprising at least one C₅ to C₇ saccharide unit substituted with at least one amino group, wherein said at least one compound is present in an amount effective to impart a durable non-permanent shape to said at least one keratinous fiber or to durably retain a non-permanent shape of said at least one keratinous fiber.

131. A composition according to claim 130, wherein said at least one amino group is chosen from unsubstituted amino groups and substituted amino groups.

132. A composition according to claim 130, wherein said at least one C₅ to C₇ saccharide unit is further substituted with at least one group different from said at least one amino group.

133. A composition according to claim 130, wherein said at least one C₅ to C₇ saccharide unit is substituted with said at least one amino group at C1 of said saccharide unit.

134. A composition according to claim 130, wherein said at least one C₅ to C₇ saccharide unit is substituted with said at least one amino group at C2 of said saccharide unit.

135. A composition to claim 130, wherein said at least one compound is chosen from C₅ monosaccharides substituted with at least one amino group, C₆ monosaccharides substituted with at least one amino group, C₇ monosaccharides substituted with at least one amino group, polymers comprising at least one C₅ monosaccharide substituted with at least one amino group, polymers comprising at least one C₆ monosaccharide substituted with at least one amino group, polymers comprising at least one C₇ monosaccharide substituted with at least one amino group, and glycoproteins comprising at least one C₅ to C₇ saccharide unit substituted with at least one amino group.

136. A composition according to claim 135, wherein said C₅ monosaccharides substituted with at least one amino group are chosen from pentosamines.

137. A composition according to claim 136, wherein said pentosamines are chosen from aldopentosamines and ketopentosamines.

138. A composition according to claim 136, wherein said pentosamines are chosen from xylosamine, arabinosamine, lyxosamine, ribosamine, ribulosamine and xylulosamine.

139. A composition according to claim 135, wherein said C₆ monosaccharides substituted with at least one amino group are chosen from hexosamines.

140. A composition according to claim 139, wherein said hexosamines are chosen from aldohexosamines and ketohexosamines.

141. A composition according to claim 140, wherein hexosamines are chosen from glucosamine, galactosamine, allosamine, altrosamine, mannosamine, gulosamine, idosamine, galactosamine, and talosamine.

142. A composition according to claim 135, wherein said C₇ monosaccharides substituted with at least one amino group are chosen from heptosamines.

143. A composition according to claim 142, wherein said heptosamines are chosen from aldoheptosamines and ketoheptosamines.

144. A composition according to claim 130, wherein said at least one compound is chosen from oligosaccharides derived from said at least one C₅ to C₇ saccharide unit substituted with at least one amino group.

145. A composition according to claim 130, wherein said at least one C₅ to C₇ saccharide unit is chosen from furanoses and derivatives thereof.

146. A composition according to claim 130, wherein said at least one C₅ to C₇ saccharide unit is chosen from derivatives of C₅ to C₇ saccharide units.

147. A composition according to claim 146, wherein said derivatives of C₅ to C₇ saccharide units are chosen from imine derivatives of C₅ to C₇ saccharide units, hemiacetal derivatives of C₅ to C₇ saccharide units, hemiketal derivatives of C₅ to C₇ saccharide units, and oxidized derivatives of C₅ to C₇ saccharide units.

148. A composition according to claim 146, wherein said derivatives of C₅ to C₇ saccharide units are further substituted with at least one group different from said at least one amino group.

149. A composition according to claim 130, wherein said at least one compound is chosen from lyxosylamine.

150. A composition according to claim 130, wherein said at least one compound is chosen from glucosamine.

151. A composition according to claim 130, wherein said at least one compound is chosen from galactosamine.

152. A composition according to claim 130, wherein said at least one compound is present in said composition in an amount ranging from 0.01% to 10% by weight relative to the total weight of the composition.

153. A composition according to claim 152, wherein said at least one compound is present in said composition in an amount ranging from 0.1% to 5% by weight relative to the total weight of the composition.

154. A composition according to claim 130, wherein said composition further comprises at least one additional'sugar, said at least one additional sugar being different from said at least one compound comprising at least one C₅ to C₇ saccharide unit substituted with at least one amino group.

155. A composition according to claim 154, wherein said at least one additional sugar is chosen from monosaccharides, oligosaccharides and polysaccharides.

156. A composition according to claim 155, wherein said monosaccharides are chosen from hexoses.

157. A composition according to claim 156, wherein said hexoses are chosen from allose, altrose, glucose, mannose, gulose, idose, galactose, talose, sorbose, psicose, fructose, and tagatose.

158. A composition according to claim 154, wherein said at least one additional sugar is present in said composition in an amount ranging from 0.01% to 10% by weight relative to the total weight of the composition.

159. A composition according to claim 158, wherein said at least one additional sugar is present in said composition in an amount ranging from 0.1% to 5% by weight relative to the total weight of the composition.

160. A composition according to claim 130, wherein said composition is in the form of a liquid, oil, paste, stick, dispersion, emulsion, lotion, gel, or cream.

161. A composition according to claim 130, wherein said at least one keratinous fiber is hair.

162. A composition according to claim 130, further comprising at least one suitable additive chosen from anionic surfactants, cationic surfactants, nonionic surfactants, amphoteric surfactants, fragrances, penetrating agents, antioxidants, sequestering agents, opacifying agents, solubilizing agents, emollients, colorants, screening agents, preserving agents, proteins, vitamins, silicones, plant oils, mineral oils, and synthetic oils.

163. A composition according to claim 130, wherein said composition is heat-activated.

164. A kit for durable non-permanent shaping of at least one keratinous fiber or for durable retention of a non-permanent shape of at least one keratinous fiber said kit comprising at least one compartment,
wherein said at least one compartment comprises a composition comprising at least one compound comprising at least one C₅ to C₇ saccharide unit substituted with at least one amino group, wherein said at least one compound is present in an amount effective to impart a durable non-permanent shape to said at least one keratinous fiber or to durably retain a non-permanent shape of said at least one keratinous fiber.

165. A kit according to claim 164, wherein said composition further comprises at least one additional sugar, said at least one additional sugar being different from said at least one compound comprising at least one C₅ to C₇ saccharide unit substituted with at least one amino group.

166. A kit according to claim 164, wherein said composition further comprises at least one film forming agent.

167. A kit according to claim 164, further comprising a second compartment comprising a composition comprising at least one film forming agent.
